(19) United States
(12) Patent Application Publication
(10) Pub. No.: US 2026/0157734 A1
(43) Pub. Date: Jun. 11, 2026

(54) ULTRASONIC DIAGNOSTIC APPARATUS, ULTRASONIC DIAGNOSTIC METHOD, AND STORAGE MEDIUM

(71) Applicant: KONICA MINOLTA, INC., Tokyo (JP)

(72) Inventors: Morio NISHIGAKI, Fujisawa-shi (JP); Mutsuhiro AKAHANE, Tokyo (JP)

(73) Assignee: KONICA MINOLTA, INC., Tokyo (JP)

(21) Appl. No.: 19/403,815

(22) Filed: Nov. 29, 2025

(30) Foreign Application Priority Data
Dec. 6, 2024 (JP) 2024-213227

(51) Int. Cl. A61B 8/00; A61B 8/08

(52) U.S. Cl. CPC A61B 8/58; A61B 8/08; A61B 8/461; A61B 8/5207

(57) ABSTRACT

An ultrasonic diagnostic apparatus includes: a transmitter/receiver that acquires a reception signal from a target site of a subject by transmission and reception of an ultrasonic wave; and a hardware processor that: calculates in real time an attenuation characteristic based on an attenuation rate at each of a plurality of positions in a predetermined area of the target site by using the reception signal acquired by the transmitter/receiver; determines in real time whether data including the calculated attenuation characteristics at the plurality of positions is satisfactory; and stores in real time the data determined to be satisfactory.

### CROSS-REFERENCE TO RELATED APPLICATIONS

The entire disclosure of Japanese Patent Application No. 2024-213227 filed on Dec. 6, 2024, is incorporated herein by reference in its entirety.

### BACKGROUND OF THE INVENTION

### Technical Field

The present disclosure relates to an ultrasonic diagnostic apparatus, an ultrasonic diagnostic method, and a storage medium.

### Description of Related Art

In the related art, in an ultrasound apparatus capable of displaying an ultrasonic image of a target site of a subject, a function is known to display a state of a property of a tissue, for example, fatty liver, from an attenuation rate of a reception signal reflected at the target site of the subject. Japanese Patent No. 7230255 describes an ultrasound apparatus that acquires an index value indicating the stability of a tissue property parameter for each subregion of a region of interest.

### SUMMARY OF THE INVENTION

By the way, in a case of calculating the amount of fat of the liver, when structures such as blood vessels in the liver are included in the irradiation area of ultrasonic waves, an accurate attenuation rate cannot be calculated. In this case, it is necessary to adjust the placement and angle of an ultrasonic probe to avoid including blood vessels in the irradiation area for the liver. However, in the related art, the position of the ultrasonic probe with respect to the target site becomes unstable due to the influence of body movement, breathing, or the like of the subject, making it difficult to acquire satisfactory data suitable for measuring fatty liver in some cases. In addition, in a case where an operator is inexperienced, even a moment at which high-quality data can be acquired may be overlooked.

Therefore, in order to solve the above-described problem, an object of the present disclosure is to provide an ultrasonic diagnostic apparatus, an ultrasonic diagnostic method, and a storage medium capable of acquiring and storing satisfactory data based on an attenuation rate of a target site of a subject in real time.

To achieve at least one of the abovementioned objects, an ultrasonic diagnostic apparatus reflecting one aspect of the present invention comprises: a transmitter/receiver that acquires a reception signal from a target site of a subject by transmission and reception of an ultrasonic wave; and a hardware processor that: calculates in real time an attenuation characteristic based on an attenuation rate at each of a plurality of positions in a predetermined area of the target site by using the reception signal acquired by the transmitter/receiver; determines in real time whether data including the calculated attenuation characteristics at the plurality of positions is satisfactory; and stores in real time the data determined to be satisfactory.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a block diagram illustrating an ultrasonic diagnostic apparatus according to an embodiment of the present disclosure;

FIG. 2 is a flowchart showing an example of an operation of the ultrasonic diagnostic apparatus in a case of acquiring data and the like relating to fatty liver of the liver according to the embodiment of the present disclosure;

FIG. 3 is a diagram illustrating an example of reception signals when reflected waves reflected by an organ or the like including the liver are received according to the embodiment of the present disclosure;

FIG. 4 is a flowchart illustrating an example of an operation of an attenuation characteristic calculator during an attenuation characteristic calculation process according to the embodiment of the present disclosure;

FIG. 5 is a diagram illustrating a frequency characteristic acquired when a first reception signal at a first depth position is frequency-converted and a frequency characteristic acquired when a second reception signal at a second depth position is frequency-converted, in the reception signal illustrated in FIG. 3; and

FIG. 6 is a diagram illustrating attenuation characteristics of a region including the first depth position and the second depth position in a depth direction of the reception signal illustrated in FIG. 3.

### DETAILED DESCRIPTION

Hereinafter, an ultrasonic diagnostic apparatus, an ultrasonic diagnostic method, and a storage medium according to one or more preferred embodiments of the present disclosure will be described in detail with reference to the accompanying drawings. However, the scope of the invention is not limited to the disclosed embodiments.

### Exemplary Configuration of Ultrasonic Diagnostic Apparatus 1

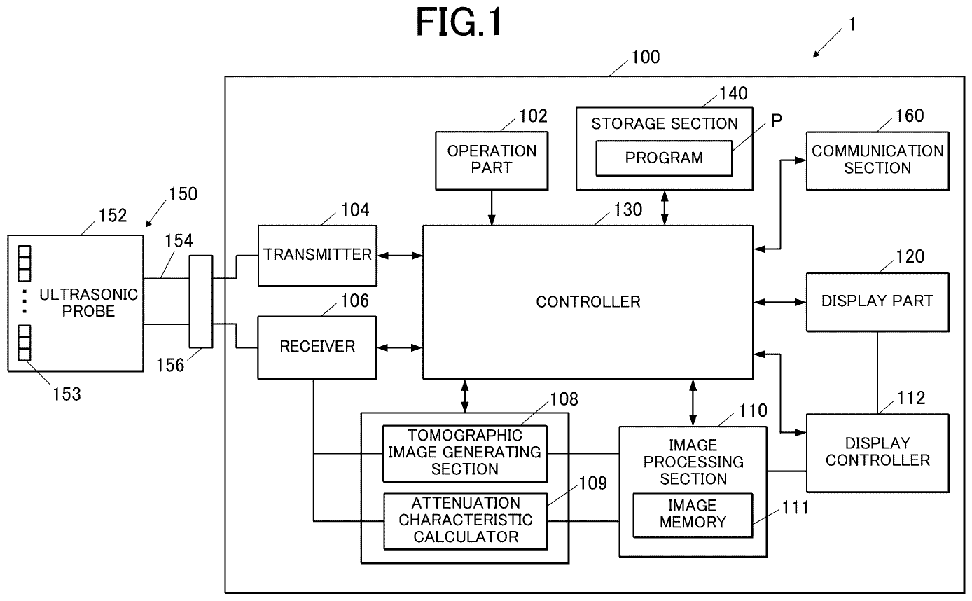

FIG. 1 is a block diagram illustrating an ultrasonic diagnostic apparatus 1 according to the present embodiment. The ultrasonic diagnostic apparatus 1 includes an apparatus body 100 and an ultrasonic probe 150 connected to the apparatus body 100. The apparatus body 100 includes an operation part 102 and a display part 120. The apparatus body 100 includes a transmitter 104, a receiver 106, a tomographic image generating section 108, an attenuation characteristic calculator 109, an image processing section 110, a display controller 112, a controller (hardware processor) 130, a storage section 140, and a communication section 160.

The operation part 102 includes, for example, at least one of a plurality of buttons, a trackball, a mouse, a touch screen combined with the display part 120, and the like. The operation part 102 receives input instructions by various user operations, converts the received input instructions into electrical signals, and outputs the electrical signals to the controller 130.

The transmitter 104 supplies a drive signal, which is an electrical signal, to the ultrasonic probe 150 under the control of the controller 130. The transmitter 104 includes, for example, a clock generation circuit, a delay circuit, and a pulse generation circuit. The clock generation circuit generates a clock signal for determining the transmission timing and transmission frequency of the drive signal. The delay circuit sets a delay time for each path provided in each probe element 153 to be described later and delays transmission of the drive signal by the set delay time. The delay circuit focuses a transmission beam constituted by ultrasonic waves. The pulse generation circuit generates a pulse signal as a drive signal at a predetermined cycle. The transmitter 104 drives, for example, a consecutive portion of a plurality of probe elements 153 to generate ultrasonic waves. The transmitter 104 performs scanning while shifting the probe elements 153 to be driven in the azimuth direction each time the ultrasonic waves are generated.

The receiver 106 receives a reception signal, which is an electrical signal, from the ultrasonic probe 150 under the control of the controller 130. The receiver 106 includes, for example, an amplifier, an A/D conversion circuit, and a phasing addition circuit. The amplifier amplifies a reception signal at a preset amplification factor for each path provided in each probe element 153. The A/D conversion circuit performs analog/digital conversion on the amplified reception signal. The phasing addition circuit gives a delay time to the A/D converted reception signal for each path provided in each probe element 153 to adjust a time phase and adds these. The phasing addition circuit generates a reception signal as sound ray data by phasing addition. Note that the receiver 106 may include an amplifier for amplifying the reception signal.

The tomographic image generating section 108 performs envelope detection processing, logarithmic compression, and the like on the reception signal supplied from the receiver 106. The tomographic image generating section 108 further adjusts at least one of the dynamic range and the gain of the reception signal to perform luminance conversion, thereby generating B-mode data. The B-mode data represents the intensity of a reception signal by luminance and is tomographic image information on a tissue of a subject.

The attenuation characteristic calculator 109 acquires satisfactory attenuation data that does not include a blood vessel or other structure by determining the variance of the attenuation characteristics at each position in a predetermined area of a target site of the subject based on the reception signal supplied from the receiver 106. The attenuation characteristic calculator 109 functions as a calculation section, a determination section, and a recording section. The calculation section calculates, in real time, an attenuation rate at each position in the depth direction in the predetermined area of the target site, which is the liver, by using the reception signal acquired by the receiver 106. The calculation section calculates an attenuation characteristic of a region including each position in real time from the calculated attenuation rate at each position in the depth direction of the predetermined area. The determination section determines in real time whether attenuation data including the attenuation characteristics is satisfactory based on the variance or the like of the attenuation characteristics in each region of the predetermined area of the liver calculated by the calculation section. The determination section determines that the attenuation data is satisfactory when the variance of the attenuation characteristics in each region of the predetermined area of the liver is within a predetermined range. The recording section performs control such that the attenuation data determined to be satisfactory by the determination section is stored in the storage section 140 or the like in real time. Note that the storage control by the recording section may be performed by the controller 130.

The image processing section 110 generates B-mode image data by, for example, performing image processing on the B-mode data output from the tomographic image generating section 108 in accordance with various image parameters that have been set. Further, the image processing section 110 generates superimposed image data by combining the generated B-mode image data with the attenuation data output from the attenuation characteristic calculator 109. The image processing section 110 includes an image memory 111 constituted by a semiconductor memory such as a DRAM. DRAM is an abbreviation for Dynamic Random Access Memory. Under the control of the controller 130, the image processing section 110 stores image data such as the B-mode image data and the superimposed image data subjected to the image processing in the image memory 111 on a frame-by-frame basis. Under the control of the controller 130, the image processing section 110 sequentially outputs the image data generated as described above to the display controller 112.

Under the control of the controller 130, the display controller 112 generates an image signal for display by performing coordinate conversion or the like on the received image data. The display controller 112 outputs the generated image signal for display to the display part 120.

Under the control of the controller 130, the display part 120 displays, on a screen, an ultrasonic image of a tissue, an organ, or the like of the subject based on the image signal for display output from the display controller 112. The ultrasonic image may be a still image or a moving image. In the present embodiment, the display part 120 can superimpose and display information on the attenuation characteristics within a predetermined area of a B-mode image. Note that the display part 120 may be a display device connected to the apparatus body 100 via, for example, a cable, a network, or the like.

The controller 130 controls the overall operation of the ultrasonic diagnostic apparatus 1. Specifically, the controller 130 controls the operations of the transmitter 104, the receiver 106, the attenuation characteristic calculator 109, the storage section 140, and the like based on various instructions input by the user via the operation part 102 and various programs and data read from the storage section 140. Furthermore, when satisfactory attenuation data is acquired by the attenuation characteristic calculator 109, the controller 130 functions as a notification section that notifies the user that the acquired attenuation data is satisfactory. As the notification means, for example, a message indicating that satisfactory attenuation data has been acquired may be displayed on the screen of the display part 120, or the user may be notified that satisfactory attenuation data has been acquired by sound or the like.

The storage section 140 includes at least one storage module, for example, an HDD, an SSD, a ROM, and a RAM. HDD is an abbreviation for Hard Disk Drive. SSD is an abbreviation for Solid State Drive. ROM is an abbreviation for Read Only Memory. RAM is an abbreviation for Random Access Memory. The storage section 140 stores, for example, system programs, application programs, and various types of data received by the communication section 160. For example, the storage section 140 stores a program P for executing processing related to ultrasound examination, saving and displaying attenuation data including the attenuation characteristics of the target site, and the like.

The communication section 160 includes, for example, an NIC, a LAN adapter, and a communication module including a receiver and a transmitter. NIC is an abbreviation for Network Interface Card. The communication section 160 communicates various kinds of data, information, and the like with an external device via a network, for example.

The ultrasonic probe 150 includes a head part 152, a cable 154, and a connector 156. The head part 152 is a portion to be pressed against the body surface of the subject. The head part 152 includes a plurality of probe elements 153 formed of piezoelectric elements. Each of the plurality of probe elements 153 transmits ultrasonic waves to the target site of the subject based on the drive signal transmitted from the apparatus body 100 and receives reflected waves reflected at the target site of the subject. For example, the plurality of probe elements 153 may be arranged in a one-dimensional array in a scanning direction or may be arranged in a two-dimensional array. The number of probe elements 153 can be suitably set. As a scanning method of the ultrasonic probe 150, a linear scanning method, a convex scanning method, a sector scanning method, or the like can be adopted.

The cable 154 has one end electrically connected to the head part 152 and the other end electrically connected to the connector 156. The connector 156 is connected to the apparatus body 100. Note that the communication between the apparatus body 100 and the ultrasonic probe 150 is not limited to wired communication using the cable 154. The communication method between the apparatus body 100 and the ultrasonic probe 150 may be wireless communication using UWB or the like. UWB is an abbreviation for Ultra-wideband.

The ultrasonic diagnostic apparatus 1 functions as a computer and includes at least one processor (hardware processor) for implementing each function of the tomographic image generating section 108, the attenuation characteristic calculator 109, the controller 130, and the like. The processor implements each function of the tomographic image generating section 108, the attenuation characteristic calculator 109, and the controller 130 by executing a program stored in the storage section 140 or a memory in a circuit of the processor. Further, in the present embodiment, the processor implements each function of the calculation section, the determination section, and the recording section of the attenuation characteristic calculator 109 by executing the above-described program. The processor includes, for example, a dedicated or general-purpose CPU or GPU. CPU is an abbreviation for Central Processing Unit. GPU is an abbreviation for Graphics Processing Unit. Further, the processor may include an application specific integrated circuit such as an ASIC or an FPGA. ASIC is an abbreviation for Application Specific Integrated Circuit. FPGA is an abbreviation for Field Programmable Gate Array. Note that each function of the tomographic image generating section 108, the attenuation characteristic calculator 109, the controller 130, and the like may be included in a single circuit. The controller 130 may include at least one of the function of the tomographic image generating section 108 and the function of the attenuation characteristic calculator 109.

### Exemplary Operation of Ultrasonic Diagnostic Apparatus 1

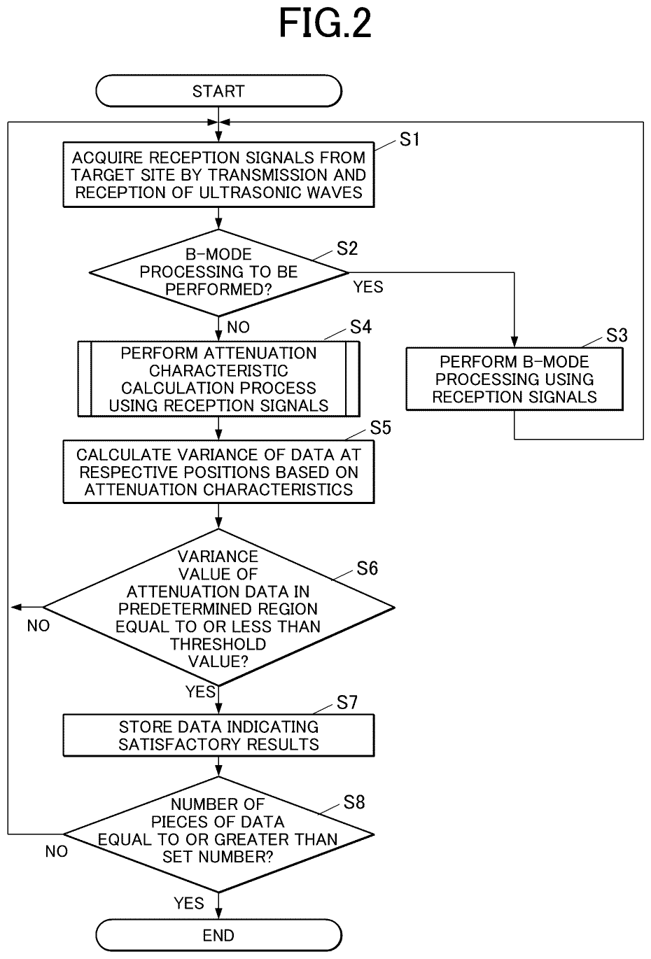

FIG. 2 is a flowchart illustrating an example of an operation of the ultrasonic diagnostic apparatus 1 in a case of acquiring and storing attenuation data and the like related to the amount of fat of the liver, which is an example of the target site according to the present embodiment. The attenuation characteristic calculator 109 or the like implements each process including a transmission and reception step, a calculation step, a determination step, and a recording step illustrated in FIG. 2 by executing the program P stored in the storage section 140 or the like.

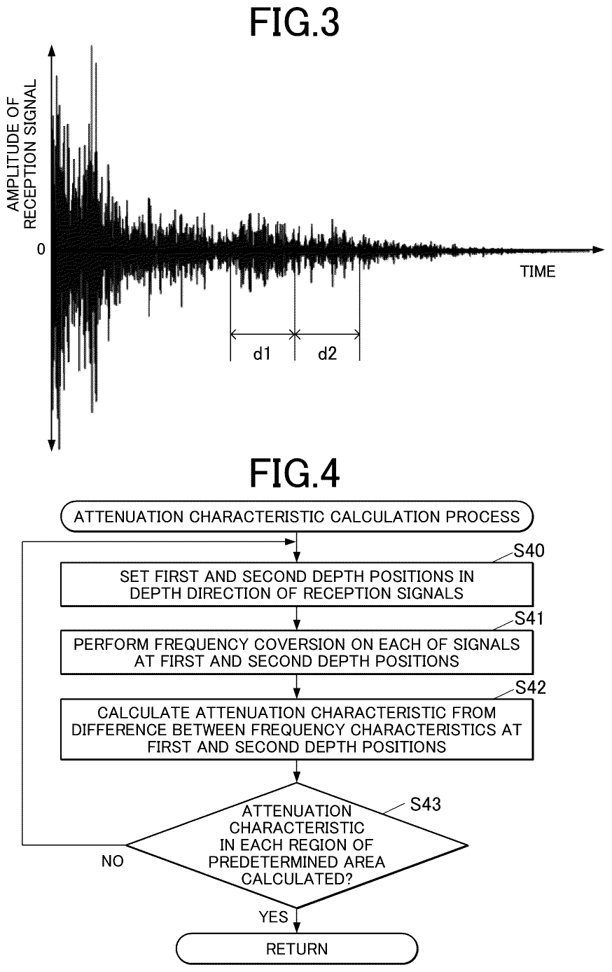

The ultrasonic probe 150 transmits ultrasonic waves toward the subject and receives reflected waves reflected by an organ such as the liver, tissue, or the like in the subject (step S1). Step S1 corresponds to an example of the transmission and reception step. FIG. 3 is a diagram illustrating an example of reception signals when the reflected waves reflected by the liver or the like are received according to the present embodiment. In FIG. 3, the vertical axis represents the amplitude of the reception signal, and the horizontal axis represents time. The left end of the horizontal axis is the generation timing of the ultrasonic waves (transmission pulses). The time on the horizontal axis is the elapsed time from the generation of the ultrasonic waves and reflects the depth direction of the liver of the subject. The ultrasonic diagnostic apparatus 1 acquires two-dimensional data by performing transmission and reception while gradually shifting the position of the plurality of probe elements 153 in the lateral direction, which is the scanning direction. The plurality of probe elements 153 forms, for example, a transducer array, and the position in the scanning direction for transmitting and receiving ultrasonic waves is changed depending on which element of the transducer array is used. The reception signals illustrated in FIG. 3 are signals transmitted and received at a predetermined position in the scanning direction of the probe elements 153. The reception signals become attenuated and smaller as the depth in the liver increases, because the propagation distance of the transmitted signals, which are ultrasonic waves, increases.

The controller 130 determines whether to perform B-mode processing (step S2). If the controller 130 determines to perform the B-mode processing, the controller 130 advances the process to step S3. On the other hand, if the controller 130 determines not to perform the B-mode processing, the controller 130 advances the process to step S4. For example, the controller 130 may determine, based on information set in advance by the user, whether to perform the B-mode processing. Note that although an example in which the B-mode processing and an attenuation characteristic calculation process are performed at different times will be described in the present embodiment, the B-mode processing and the attenuation characteristic calculation process may be performed in parallel.

The tomographic image generating section 108 performs processing, such as envelope detection processing, logarithmic compression, dynamic range and gain, on the reception signals output from the receiver 106 to perform luminance conversion, thereby generating B-mode data (step S3). The tomographic image generating section 108 outputs the generated B-mode data to the image processing section 110, and the controller 130 returns the process to step S1. Note that when only the B-mode processing is performed, an ultrasonic image may be displayed on the display part 120 based on B-mode image data generated by the image processing section 110, and the series of processing may be ended.

On the other hand, if the controller 130 determines not to perform the B-mode processing, the controller 130 controls the attenuation characteristic calculator 109 to perform the attenuation characteristic calculation process. The attenuation characteristic calculator 109 performs the attenuation characteristic calculation process for calculating attenuation characteristics and the like at a predetermined position in a predetermined area of the liver by using the reception signals output from the receiver 106 (step S4). Step S4 corresponds to an example of the calculation step. The controller 130 advances the process to the subroutine illustrated in FIG. 4 in order for the attenuation characteristic calculator 109 to perform the attenuation characteristic calculation process.

FIG. 4 is a flowchart illustrating an example of the operation of the attenuation characteristic calculator 109 during the attenuation characteristic calculation process in step S4. The attenuation characteristic calculator 109 sets each of a first depth position d1 at a certain depth and a second depth position d2 deeper than the first depth position d1 in the reception signals output from the receiver 106, as illustrated in FIG. 3 (step S40).

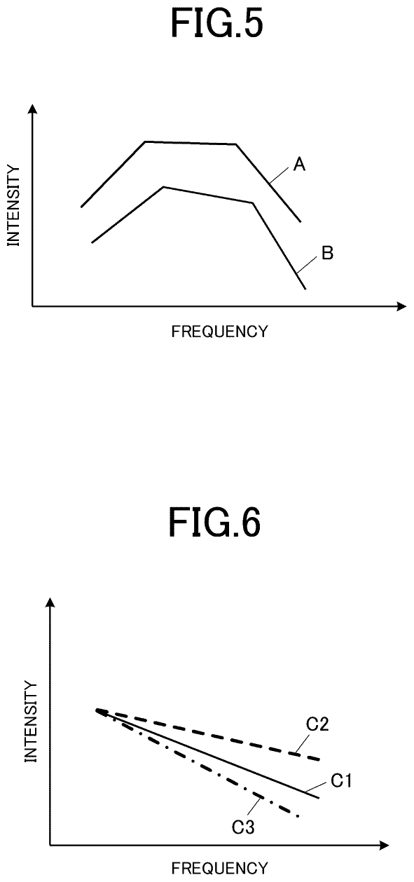

The attenuation characteristic calculator 109 performs frequency conversion on each of a first signal at the first depth position d1 and a second signal at the second depth position d2 (step S41). Examples of the frequency conversion include a Fourier transform and a wavelet transform. FIG. 5 is a diagram illustrating, in the reception signals illustrated in FIG. 3, a frequency characteristic A obtained when the first signal at the first depth position d1 is frequency-converted and a frequency characteristic B obtained when the second signal at the second depth position d2 is frequency-converted. In FIG. 5, the vertical axis represents intensity, and the horizontal axis represents frequency. The attenuation of the reception signals in the liver of the subject is greater at higher frequencies. The attenuation of the frequency characteristic B at the second depth position d2, which is deeper, is greater than the attenuation of the frequency characteristic A at the first depth position d1.

The attenuation characteristic calculator 109 calculates the attenuation characteristic in a local region including the first depth position d1 and the second depth position d2 from the difference between the frequency characteristic A at the first depth position d1 and the frequency characteristic B at the second depth position d2 (step S42). FIG. 6 is a diagram illustrating attenuation characteristics in the local region near the first depth position d1 and the second depth position d2 in the depth direction of the reception signals illustrated in FIG. 3. In FIG. 6, the vertical axis represents intensity, and the horizontal axis represents frequency. The attenuation characteristic indicates the amount of attenuation of the ultrasonic waves when the transmission signals are propagated in the local region from the first depth position d1 to the second depth position d2. The attenuation characteristic is expressed, for example, as a linear function having a constant slope. The linear function includes a curve that can be approximated by a straight line. For example, in a case where the local region is mainly composed of only liver cells, the attenuation of the reception signals becomes uniform, and the attenuation characteristic becomes an attenuation characteristic C1 as indicated by the solid line in FIG. 6. In a case where the local region is composed of blood vessels and liver cells, the attenuation is smaller than that in the case of only liver cells, i.e., normal liver, and the attenuation characteristic becomes an attenuation characteristic C2 as indicated by the dotted line in FIG. 6. In addition, in a case where the local region is mainly composed of fatty liver, the attenuation of the reception signals is greater due to the occurrence of ultrasonic wave scattering by fat than that in the case of only normal liver, and the attenuation characteristic becomes an attenuation characteristic C3 as indicated by the one-dot chain line in FIG. 6. Note that in the present embodiment, unlike cirrhosis, it is assumed that the degree of lesion does not vary depending on the site of the liver.

The attenuation characteristic calculator 109 determines whether the attenuation characteristic in each local region of the predetermined area of the liver has been calculated (step S43). If the attenuation characteristic calculator 109 determines that the attenuation characteristic in each local region of the predetermined area of the liver has been calculated, the process proceeds to step S5 illustrated in FIG. 2.

On the other hand, in step S43, if the attenuation characteristic calculator 109 determines that the attenuation characteristic in each local region of the predetermined area of the liver has not been calculated, the process returns to step S40. In this case, the attenuation characteristic calculator 109 sets a depth position different from the first depth position d1 and the second depth position d2 in the reception signals illustrated in FIG. 3 and calculates the attenuation characteristic and the like in a local region including the set depth position. Furthermore, the attenuation characteristic calculator 109 sets a predetermined depth position in reception signals at a position shifted in the scanning direction (lateral direction) within a predetermined area and calculates the attenuation characteristic and the like in a local region including the set predetermined depth position. In this way, the attenuation characteristic calculator 109 acquires the attenuation characteristics in a plurality of local regions in a predetermined area of a scan region of the liver. Hereinafter, the slope of the attenuation characteristic may be referred to as an attenuation parameter. The unit of the attenuation parameter is [dB/cm/MHz], indicating how many dB a signal attenuates per frequency of 1 MHz during propagation over a unit distance.

As illustrated in FIG. 2, the attenuation characteristic calculator 109 calculates a variance value of the calculated attenuation parameters at the respective depth positions in the predetermined area using a statistical method (step S5). Note that as the statistical method, a standard deviation or the like may be used in addition to the variance.

The attenuation characteristic calculator 109 compares the calculated variance value of the plurality of attenuation parameters with a preset threshold value to determine whether the variance value is equal to or less than the threshold value (step S6). Step S6 corresponds to an example of the determination step. For example, the threshold value may be determined using data collected in clinical practice in consideration of variance values when a structure such as a blood vessel is included, when the liver is normal, and when fatty liver is present. If the variance value is equal to or less than the threshold value, the attenuation characteristic calculator 109 determines that the attenuation data including the plurality of attenuation parameters in the predetermined area is satisfactory. This is because, when normal liver and fatty liver are included in the liver, although the attenuation amounts are different from each other, the attenuation parameters, which represent the state of the liver, fall within a certain range. Therefore, the variation of the attenuation parameters becomes small, leading to a smaller variance value.

On the other hand, if the variance value exceeds the threshold value, the attenuation characteristic calculator 109 determines that the attenuation data including the plurality of attenuation parameters in the predetermined area is not satisfactory. For example, when a structure, such as a blood vessel, and a liver cell are included in the scan region of the liver, the attenuation amount of the blood vessel and the attenuation amount of the liver cell are greatly different from each other, and the attenuation parameters, which represent the state of the liver, do not fall within a certain range. Therefore, the variation of the attenuation parameters becomes large, leading to a greater variance value. In this case, since it is considered that there is a problem in the measurement site and/or measurement conditions, the process returns to step S1, transmission and reception of ultrasonic waves to and from the target site of the subject are performed to collect attenuation data again.

The attenuation characteristic calculator 109 stores the attenuation data determined to be satisfactory in the storage section 140 or the like in real time (step S7). Step S7 corresponds to an example of the recording step. For example, the attenuation data may be image data in which each depth position in the predetermined area of the liver is colored according to the amount of fat based on the attenuation parameters by using a color map associated with the respective attenuation parameters. In addition, the attenuation characteristic calculator 109 may store quantified numerical values of the plurality of attenuation parameters in the predetermined area of the liver in the storage section 140 in real time. Furthermore, the image processing section 110 or the like may generate the attenuation data. The various data may be stored in the image memory 111 of the image processing section 110.

The attenuation characteristic calculator 109 determines whether the number of pieces of attenuation data indicating satisfactory results has reached a preset number (step S8). If the attenuation characteristic calculator 109 determines that the number of pieces of attenuation data indicating satisfactory results has reached the preset reference number, the attenuation characteristic calculator 109 ends the series of scans by ending the scanning of the next frame by the transmitter 104 or the like. In this case, the attenuation characteristic calculator 109 may calculate an average value of the attenuation parameters in the respective local regions from the plurality of pieces of attenuation data indicating satisfactory results. On the other hand, if the attenuation characteristic calculator 109 determines that the number of pieces of attenuation data indicating satisfactory results has not reached the preset reference number, the process returns to step S1. In this case, for example, the attenuation characteristic calculator 109 scans the next frame and calculates the attenuation rate, the attenuation characteristic, the variance, and the like of the acquired reception signals at each depth position in the predetermined area. Note that the controller 130 may simultaneously perform the scanning of the next frame and the storage of the attenuation data described above. In addition, the controller 130 may determine the above-described reference number based on the magnitude of variance of the plurality of attenuation parameters. For example, when the variance value of the attenuation parameters tends to be large, the reference number may be increased.

Note that the image processing section 110 may generate a superimposed image by combining the generated B-mode image with the attenuation data generated by the attenuation characteristic calculator 109. In this case, the controller 130 causes the superimposed image generated by the image processing section 110 to be displayed on the screen of the display part 120. Thus, the predetermined area of the liver can be colored and displayed according to the amount of fat, and the spatial distribution of the attenuation parameters can be accurately grasped.

In the present embodiment, the calculation section calculates attenuation rates and attenuation parameters in a predetermined area of the liver in real time. Next, the determination section determines in real time that attenuation data including the calculated attenuation parameters in which the variance or variation of the calculated attenuation parameters in the predetermined area of the liver is within a certain range is satisfactory. In this step, attenuation data in which a structure such as a blood vessel is included has a variance outside the certain range and thus is determined to be unsatisfactory and automatically excluded. Next, the recording section stores the attenuation data including the attenuation characteristics determined to be satisfactory in the storage section 140 or the like in real time. That is, according to the present embodiment, at the moment when attenuation data of satisfactory quality in which a structure such as a blood vessel is not included is acquired, the attenuation data can be stored in the storage section 140 or the like. Thus, a user such as a physician can automatically acquire attenuation data of satisfactory quality without needing to be conscious of the position of the ultrasonic probe relative to the target site of the subject, occurrence of body motion of the subject, the physique of the subject, the properties of body tissues, and the like. As a result, according to the present embodiment, since satisfactory attenuation data can be efficiently stored, it is possible to reduce the operation load of the ultrasonic probe or the like by the user.

Although the preferred embodiment of the present disclosure has been described in detail with reference to the accompanying drawings, the technical scope of the present disclosure is not limited to such examples. Furthermore, those to which various modification examples and improvements have been applied naturally belong to the technical scope of the present disclosure within the category of the technical idea described in the scope of the claims of those skilled in the art.

Although embodiments of the present invention have been described and illustrated in detail, the disclosed embodiments are made for purposes of illustration and example only and not limitation. The scope of the present invention should be interpreted by terms of the appended claims.

What is claimed is:

1. An ultrasonic diagnostic apparatus comprising:
a transmitter/receiver that acquires a reception signal from a target site of a subject by transmission and reception of an ultrasonic wave; and
a hardware processor that:
calculates in real time an attenuation characteristic based on an attenuation rate at each of a plurality of positions in a predetermined area of the target site by using the reception signal acquired by the transmitter/receiver;
determines in real time whether data including the calculated attenuation characteristics at the plurality of positions is satisfactory; and
stores in real time the data determined to be satisfactory.

2. The ultrasonic diagnostic apparatus according to claim 1, wherein the hardware processor determines that the data is satisfactory when a variance of the attenuation characteristics at the plurality of positions is equal to or less than a preset threshold value.

3. The ultrasonic diagnostic apparatus according to claim 1, wherein storage of the data by the hardware processor and scanning to acquire the reception signal for a next frame by the transmitter/receiver are performed simultaneously.

4. The ultrasonic diagnostic apparatus according to claim 1, wherein the hardware processor makes a notification when the hardware processor determines that the data is satisfactory.

5. The ultrasonic diagnostic apparatus according to claim 1, wherein when having acquired a preset number of pieces of the data determined to be satisfactory, the hardware processor causes the transmitter/receiver to end scanning of a next frame.

6. The ultrasonic diagnostic apparatus according to claim 5, wherein the hardware processor determines the number based on a magnitude of a variance of the data.

7. An ultrasonic diagnostic method comprising:
acquiring a reception signal from a target site of a subject by transmission and reception of an ultrasonic wave;
calculating in real time an attenuation characteristic based on an attenuation rate at each of a plurality of positions in a predetermined area of the target site by using the reception signal acquired in the acquiring;
determining in real time whether data including the attenuation characteristics at the plurality of positions calculated in the calculating is satisfactory; and
storing in real time the data determined to be satisfactory in the determining.

8. A non-transitory computer-readable storage medium storing a program that causes a computer to function as:
a transmitter/receiver that acquires a reception signal from a target site of a subject by transmission and reception of an ultrasonic wave; and
an attenuation characteristic calculator that:
calculates in real time an attenuation characteristic based on an attenuation rate at each of a plurality of positions in a predetermined area of the target site by using the reception signal acquired by the transmitter/receiver;
determines in real time whether data including the calculated attenuation characteristics at the plurality of positions is satisfactory; and
stores in real time the data determined to be satisfactory.
